(19) United States
(12) Patent Application Publication
(10) Pub. No.: US 2006/0051868 A1
(43) Pub. Date: Mar. 9, 2006

(54) Methods for increasing conifer somatic embryo initiation, capture, and multiplication

(76) Inventors: Gerald S. Pullman, Alpharetta, GA (US); Gary F. Peter, Atlanta, GA (US)

Correspondence Address: TROUTMAN SANDERS LLP, 600 PEACHTREE STREET , NE, ATLANTA, GA, 30308, US

(21) Appl. No.: 11/265,474

(22) Filed: Nov. 1, 2005

Related U.S. Application Data
(62) Division of application No. 10/076,633, filed on Feb. 19, 2002, now abandoned.
(60) Provisional application No. 60/270,165, filed on Feb. 22, 2001.

(51) Int. Cl. C12N 5/00; C12N 5/02

(52) U.S. Cl. 435/422

(57) ABSTRACT

The present invention provides methods for initiating, capturing, maintaining and multiplying embryogenic cultures of coniferous plants. Methods include the use of novel media compositions containing folic acid, biotin, pH buffers, gibberellin inhibitors and gas-tight environment treatments to improve the frequency of embryogenic tissue initiation, capture, maintenance and multiplication. The methods are well suited for initiating embryogenic cultures in recalcitrant conifer varieties. The method is also well suited for producing somatic embryos that can be further cultured to produce large numbers of plants. Further, the invention provides novel methods that may be used to enhance somatic embryogenesis in a broad range of species.

### CROSS-REFERENCE TO RELATED APPLICATIONS

This patent application claims benefit of priority of provisional application U.S. Ser. No. 66/270,165, filed Feb. 22, 2001.

### FIELD OF THE INVENTION

The present invention relates to the use of somatic embryogenesis to produce genetically identical mature organisms at high rates; The present invention further relates to the use of conifer somatic embryos. The invention relates to the development of culture conditions for improved initiation and multiplication rates of conifer embryogenic cultures. Aspects of the invention relating to culture modifications include the changes to vitamins levels, buffering, gas exchange rates, and atmospheric pressure.

### BACKGROUND OF THE INVENTION

Somatic embryogenesis is widely used in a variety of plant species to produce multiple copies of genetically identical organisms. In some species, somatic embryogenesis is used to propagate desirable plant genotypes. In many crop species, somatic embryogenesis is used to propagate whole transformed plants from tissues that have been genetically altered. Somatic embryogenesis protocols have been developed for the reproduction of conifers. For example, U.S. Pat. Nos. 4,957,866, 5,034,326, 5,036,007, 5,236,841, 5,413,930, 5,491,090, and 5,506,136, herein incorporated by reference, describe various methods and media for conifer embryogenesis.

Somatic embryogenesis is a multi-step process by which an individual plant is clonally propagated. Tissue from the parent plant is induced to form embryos. Subsequent culturing steps are then performed to induce the embryos to mature into plantlets. These small plants are capable of growing into mature plants, each of which is genetically identical to the parent. Using this process, an individual plant with a desirable genotype can be efficiently reproduced hundreds or thousands of times.

In conifers, somatic embryogenesis begins with “initiation,” the initial formation of embryogenic cultures. Embryogenic cultures contain one or more immature somatic embryos. Initiation is traditionally followed by a maintenance and multiplication phase in which large numbers of clones of the initiated somatic embryo(s) are produced. The embryos produced in the maintenance and multiplication step(s) are then further cultured on a development or maturation media. This development media induces the immature embryos to mature into late-stage embryos capable of germination. These mature embryos are often placed on a germination medium where they germinate to form plantlets. The plantlets can be grown and acclimated to the point that they are capable of being planted in soil. Subsequently these small plantlets will grow into mature trees.

Many conifer species contain recalcitrant genotypes not readily regenerated. The commercially important Loblolly pine, for example, is generally difficult to propagate by somatic embryogenesis (Becwar et al., Can. J. For. Res. 20:810 (1990), Jain et. al., Plant Sci. 65:233-241 (1989)). Further, genetic transformation techniques used to generate transgenic conifers, such as Agrobacterium-mediated gene transfer, electroporation, and particle bombardment, can damage plant cells. Damaged cells are less likely to regenerate into whole plants. Thus, there is a need in the art for methods and compositions that improve the efficiency of somatic embryogenesis, including somatic embryogenesis in conifers.

Initiation of Embryogenic Cultures

Somatic embryogenesis begins with the “initiation” step. Initiation starts with the selection of a suitable explant, that is any plant cell, tissue or organ capable of forming an embryogenic culture. A typical explant in conifer somatic embryogenesis is the megagametophyte, also called the ovule or the female gametophyte, which is extracted from a pollinated female cone and which may. contain single or multiple zygotic seed embryos. One or more cells of the explant are then induced to proliferate into a tissue mass containing at least one early stage somatic embryo. The successful establishment of such a culture is known as initiation.

The cultures can be initiated from several types of explants. Most commonly, conifer embryogenic cultures are initiated from zygotic embryos or zygotic embryogenic tissues found. in seeds. When intact conifer megagametophytes are used as the explant, the somatic embryogenesis process comprises a distinct step called extrusion. Extrusion is the process in which a mass of embryogenic tissue is extruded from the micropylar end of the megagametophyte when it is placed on or in a suitable culture media.

Successfully initiating embryogenic cultures require proper medium and culturing conditions. In conifers, an embryogenic culture is successfully initiated when the zygotic embryo or zygotic embryogenic tissue mass, which has been either extruded or physically removed from a megagametophyte, undergoes division and proliferation. A successfully initiated culture consists of a whitish translucent mucilaginous tissue mass that contains pre-embryonal cells, filamentous suspensor-like cells, and early stage somatic embryos. In a successfully initiated culture, new somatic embryos can often be seen growing directly from older zygotic embryos. Visualization of initiation is aided by the fact that zygotic embryos, as well as extruded tissues, often become brown while initiated tissues are whiter and more translucent. Initiated cultures contain from one to dozens of somatic embryos. Initiation is considered successful when at least one somatic embryo is visible. The appearance of at least two somatic embryos provides a useful confirmation of successful initiation.

Maintenance and Multiplication of Embryogenic Cultures

In conifer somatic embryogenesis, initiation is generally followed by one or more “maintenance” and “multiplication” steps. Some protocols consist of several maintenance and multiplication steps, each with its own media and culturing conditions. Other protocols utilize a single maintenance and multiplication step. “Maintenance” refers to the preservation of cultures by keeping them viable through continuous growth. “Multiplication” refers to the proliferation of such cultures to provide numerous somatic embryos. Regardless of whether the steps are separate or combined into a single culturing step, this phase of somatic embryogenesis requires that previously initiated embryogenic cultures survive and proliferate, continuously producing viable immature somatic embryos. This requires the proper media and culture conditions.

Various methods of maintaining and multiplying cultures have been described. In some protocols, initiated cultures are grown using a single media composition prior to transfer to a development and maturation media. For example, Gupta et al., U.S. Pat. No. 5,563,061, describe a method for conifer somatic embryogenesis wherein initiated embryogenic cultures are transferred to a single maintenance and multiplication media where they are cultured to increase the number and size of the embryos. After culturing on this maintenance and multiplication media, the somatic embryos are transferred to a maturation and development media. Notably, however, the development, maturation and germination steps of somatic embryogenesis are not 100% efficient. In order for this protocol to be used effectively, a large number of somatic embryos must be produced.

Handley et al., U.S. Pat. No. 5,491,090, describe methods of somatic embryogenesis where initiated cultures are transferred to a liquid maintenance culture. Handley et al. additionally describe a protocol where embryogenic cultures are initiated on a first media, transferred to a second media, and thereafter transferred to a liquid media for rapid multiplication. The use of a liquid culture is especially advantageous in that handling liquid cultures is much less labor-intensive than manipulating cultures on solid media. This ease of handling makes liquid culture practical for the large scale production of seedlings via somatic embryogenesis. Large amounts of somatic embryos can be efficiently produced in liquid cultures, with cultures multiplying as much as 2-6 times weekly. However, the inventors have observed that tissue transferred to liquid culture often does not survive and proliferate. There is a need in the art for methods that will increase the efficiency of embryogenic culture multiplication using liquid media.

The success of the maintenance and multiplication step is vital for the ultimate generation of-plantlets. Embryogenic cultures, once successfully initiated, must proliferate and supply sufficient numbers of somatic embryos such that a reasonable number will ultimately be converted to plantlets. There is a need in the art for methods which improve the growth of embryogenic cultures such that large numbers of somatic embryos may be produced. Further, methods which improve the growth of embryogenic cultures will save time and reduce production costs by more rapidly producing the desired numbers of embryos. Additionally, efficient methods of maintaining. and multiplying embryogenic cultures can reduce the number of culturing steps, greatly reducing the time and expenses of the propagation process.

### SUMMARY OF THE INVENTION

The present invention provides methods for producing multiple copies of genetically identical conifer plants. The present invention provides several methods for increasing the efficiency of conifer embryogenesis. As such, the invention further encompasses methods for regenerating recalcitrant genotypes. Embryogenic tissue of the invention includes both somatic and zygotic. More specifically, invention encompasses methods for initiating, capturing, maintaining and multiplying embryogenic cultures.

The invention encompasses methods for increasing the efficiency of conifer initiation. More particularly, the present invention provides methods that include the use of media compositions to improve overall initiation frequency. The preferred media compositions of the invention contain biotin, folic acid, pH buffers, and gibberellin inhibitors. As such, included in the present invention are the culture media for increasing the efficiency of initiation. The invention further provides methods of increasing the efficiency of initiating conifer embryogenic cultures by culturing the tissue in a closed container. Additional embodiments of the invention are methods of initiating embryogenic cultures in a closed containers with varying degrees of gas exchange.

The present invention encompasses methods for growing previously initiated conifer embryogenic tissues. More particularly, the present invention provides methods that include use of media compositions to increase growth of an embryogenic culture. The preferred media compositions of the invention contain biotin, folic acid, pH buffers, gibberellin inhibitors. As such, included in the present invention are the culture media for increasing growth of previously initiated conifer embryogenic tissue. A preferred media composition of the invention for the method of improving culture capture includes abscisic acid. One embodiment of the present invention include the use of a multiplication media. The invention further provides methods of improving conifer embryogenic growth by culturing tissue in a closed container. Additional embodiments of the invention are methods for growing previously initiated conifer embryogenic tissues in a closed container with varying degrees of gas exchange.

### DETAILED DESCRIPTION OF THE INVENTION

The present invention provides several methods for increasing the efficiency of conifer somatic embryogenesis. More specifically, the invention provides methods for increasing the efficiency of the culture initiation, maintenance and multiplication phases of somatic embryogenesis. To best appreciate the scope of the invention, it is important to keep in mind the distinction between promoting culture initiation to form a tissue mass, and promoting the subsequent growth and multiplication of the initiated tissue mass. The methods of the invention are applicable to both of these critical processes.

Many embodiments of the invention comprises the “use” of various media compositions. By “use,” the inventors refer to the process of placing cultured tissues on or in a liquid, semi-solid or solid culture medium. The invention is not limited to media of any consistency and encompassed the use of media ranging from liquid to solid.

Culture Initiation

The goal of culture initiation is to create embryogenic tissue that is capable of proliferation. Culture initiation begins when cells from the explant are induced to divide and differentiate into embryogenic cells. These cells must retain the ability to continue replicating. Methods of the invention increase the frequency of successful initiation overall. It is not presently understood how the various embodiments of the invention promote initiation. The factors disclosed below may promote differentiation of cells into those with an embryogenic identity. Alternatively, these factors may increase the robustness of newly formed embryogenic cells such that they more frequently survive or more vigorously grow. Regardless of how the various embodiments of the invention work, it is an object of the invention to increase the likelihood of successful initiation.

Methods of Improving the Efficiency of Embryogenic Culture Maintenance and Multiplication

Another object of the invention is to improve the efficiency of the maintenance and multiplication steps in conifer somatic embryogenesis. These steps require the vigorous proliferation of embryogenic cultures and the production of numerous somatic embryos. The invention fulfills this objective by providing methods which increase the growth of embryogenic tissues. By “growth,” the inventors refer to the survival and proliferation of previously initiated embryogenic cultures. “Growth” can be characterized by any increase in culture size (i.e. area covered on a petri dish), mass, or number of embryos. The “maintenance and multiplication step” refers to the process of growing an embryogenic culture that has been previously initiated in order to provide somatic embryos.

A “previously initiated embryogenic culture” comprises any embryogenic tissue capable of proliferation. Such a culture may consist of an embryogenic mass containing somatic embryos, early proembryos and suspensor cells. Such a culture may comprise one or more individual somatic embryos. A previously initiated culture may consist of embryogenic tissues recently formed on an initiation medium. Alternatively, a previously initiated culture can be a subculture of tissues growing on a maintenance media. Alternatively, a previously initiated culture could consist of stored embryogenic tissues or somatic embryos, such as cryopreserved embryos.

The invention encompasses the use of novel media constituents and culturing conditions in order to increase the growth of embryogenic cultures. The invention contemplates that previously initiated embryogenic cultures will be placed on or in a maintenance media and cultured so as to induce growth. The methods provided by the invention increase the efficiency of the maintenance and multiplication step by providing one or more of the following advantages, more embryos are produced, embryos are produced more rapidly, and a greater number of previously initiated cultures survive to be successfully regenerated into plantlets.

Improving the Efficiency of Liquid Media Culture Multiplication

Further, the invention provides methods which improve the efficacy of using liquid multiplication media. The use of liquid multiplication media, for example as described in Example 7 below, can potentially provide very large numbers of somatic embryos. Nonetheless, the inventors have observed that many initiated cultures do not survive when transferred to liquid multiplication media. For example, the inventors have observed that on average, only 13-33% of initiated cultures of Loblolly pine transferred to a multiplication media will successfully survive and proliferate (data not shown). An indicator of successful proliferation is the doubling in mass of a transferred initiated culture. Hereafter, a culture that survives and proliferates in a liquid multiplication media will be referred to as having been “captured.”

The inventors have further observed that the frequency of capture is strongly related to the starting weight of the tissue transferred to the multiplication media. In experiments on Loblolly pine, the majority of nine week-old initiated cultures weighed less than 150 mg and had an average survivorship of about 15%. However, initiated cultures with a mass of 150 mg or greater survived about 80% of the time, and those with a tissue mass of 200 mg or greater survived 88% of the time. Thus, the efficiency of using liquid multiplication media is poor at least in part because many of the initiated cultures are too small to survive and proliferate when transferred to liquid multiplication media.

The invention provides methods which increase the frequency of embryogenic culture capture. Utilizing the methods of the invention one can produce an increased proportion of larger cultures, for example, those with a mass of 200 mg or greater. These larger and more robust embryogenic cultures are more likely to survive transfer to. a liquid multiplication media.

Species and Tissues Amenable to the Invention

The present invention is generally suitable for reproducing woody gymnosperms of the order Coniferales. The invention is well suited for propagating trees from species within the families Pinaceae, Cupressaceae, and. Taxodiaceae. All species within the genera Abies, Pinus, Picea, Tsuga, Pseudotsuga, Thuja, Juniperis, Larix, Taxus and Sequoia are amenable to multiplication using the disclosed methods and compositions. For example, the invention is well suited to the Pinus species such as Pinus taeda (loblolly pine), Pinus elliottii (slash pine), Pinus palustris (longleaf pine), Pinus serotina (pond pine), Pinus radiata (Monterey pine), and Pinus rigida (pitch pine), as well as other species. In addition, the invention is applicable to hybrids (i.e., interspecies hybrids) of the above mentioned pines, including crosses between Pinus_{—}rigida and Pinus taeda, crosses between Pinus serotina and Pinus taeda, and reciprocal crosses.

For initiating embryogenic cultures, any conifer tissue explant capable of being employed for somatic embryogenesis is suitable for use in the present invention. A number of explant sources have been used successfully in somatic embryogenesis. These include, but are not limited to, tissue from cotyledons, hypocotyls, epicotyls, buds, meristematic centers from buds or roots, tissues extruded from megagametophytes, and seed embryos. One practicing the invention can use an immature whole megagametophyte containing zygotic embryos or an isolated immature dominant zygotic embryo as. the explant. Zygotic embryos removed from seeds can be used. These may or may not include the surrounding gametophyte.

Increasing Embryogenic Culture Initiation and Growth with Folic Acid and Biotin

The Inventors have discovered that incorporating the B-vitamins folic acid and biotin into culture. media can increase the efficiency of conifer embryogenic culture initiation. Additionally, when these B-vitamins are added to maintenance or multiplication media, embryogenic culture growth is promoted.

The invention comprises the use of both folic acid and biotin, which may be in any form, including free acids, salts, complexes, etc. Folic acid is also known as folate. The folates are a group of heterocyclic compounds based on pteroic acid. Biotin is a water-soluble vitamin, generally classified as a B-complex vitamin. Its biochemical function is as a covalently-bound cofactor on a family of enzymes that catalyze reactions in a variety of crucial metabolic processes.

Improved initiation and growth can be attained using a media that contains either folic acid or biotin at a concentration of between about 0.001 to 10, 0.01 to 1, or 0.1 to 0.5 ppm, or any concentration subsumed within those ranges. Media may also contain both folic acid and biotin. When used in combination each may be used at a concentration of about 0.001 to 10, 0.005 to 1.0, or 0.01 to 0.01 ppm, or any concentration subsumed within those ranges.

Increasing Embryogenic Culture Initiation and Growth with Gibberellin Inhibitors

Gibberellins are plant growth regulators generally involved in growth and cell expansion. Surprisingly, the inventors have discovered that gibberellin inhibitors, can increase the efficiency of embryogenic culture initiation in conifers. These compounds can be incorporated into culture media to increase the efficiency of conifer embryogenic culture initiation and to promote the growth of embryogenic cultures. Alternatively, in initiation, explants can be pre-treated with gibberellin inhibitors prior to culturing on a media free of gibberellin inhibitors.

A gibberellin inhibitor may affect any aspect of gibberellin biosynthesis, binding activity, or the transmission of gibberellin induced signals in plants. In one embodiment, the gibberellin inhibitor comprises paclobutrazol. In another embodiment, the gibberellin inhibitors comprise chlormequat-Cl, flurprimidol, or succinic acid-DH. Other gibberellin-inhibiting compounds useful in the practice of the invention are listed in Table 1 and in Rademacher, Annual Review of Plant Physiology and Plant Molecular Biology, 51:501-31, 2000, which is hereby incorporated by reference. Media may contain a gibberellin inhibitor at a concentration of between 0.001 to 500, 0.01 to 50, or 0.1 to 5.0 ppm, or any concentration subsumed within those ranges. Media may contain a combinations of gibberellin inhibitors each of which may be present in this range of concentrations.

TABLE 1
Compounds that inhibit the action or synthesis of gibberellins.

| Class of | |
|---|---|
| compounds | Representative examples |
| Onium Compounds | Chlormequat-chloride, |
| | Mepiquat-chloride, |
| | AMO-1618, and |
| | Chlorphonium-chloride |
| Nitrogen-Containing | pyrimidines: ancymidol, flurprimidol |
| Heterocycles | norbornanodiazetins: tetcyclacis |
| | triazole-type compounds: paclobutrazol, |
| | uniconazole-P, triapenthenol |
| | 4-substituted pyridines: inabenfide |
| | imidazoles: 1-n-decylimidazole, 1-geranylimidazole |
| | HOE 074784 |
| | side activity triazole-types: triadimenol, triadimefon, |
| | ipconazole, tebuconazole, metconazole. |
| Mimics of 2- | acylcyclohexanediones: prohexadione-calcium, |
| Oxoglutaric | trinexapac-ethyl, LAB 198 999 |
| | diaminozide |
| 16, 17-Dihydro- | 16, 17-Dihydro-GA₅ and derivatives such |
| Gibberellic acids | as exo-16, 17-Dihydro-GA₅-13-acetate |

Increasing Embryogenic Culture Initiation and Growth by Maintaining a Desirable pH

Culturing conditions, including pH, strongly influence the growth of cultured plant tissues. The optimal pH for culturing plant cells varies with species and the stage of culturing. For example, the initiation of conifer embryogenic cultures is enhanced when media pH is around 4.5 to 6. Practitioners of conifer somatic embryogenesis formulate media with a pH suited to the particular types of cells being cultured. Nevertheless, the metabolic activity of tissues in these cultures will often acidify the culture media, causing media pH to decrease below the desirable range. The inventors have observed that such overly acidic conditions may have an inhibitory effect on the initiation or the growth of embryogenic cultures.

The inventors have discovered that maintaining a desirable media pH can increase initiation frequency and promote embryogenic culture growth. “Maintaining a desirable pH” is hereby defined as using any method which tends to stabilize pH at a desirable level despite metabolic activity that tends to unfavorably change pH. Alternatively, maintaining a desirable pH consists of periodically treating the culture media to counteract unfavorable pH changes which result from metabolic activity. A “desirable pH” is hereby defined as that which is favorable for a particular stage of somatic embryogenesis. For example, in culture initiation, a desirable pH is that which promotes the formation of embryogenic cultures. In culture maintenance and multiplication, a desirable pH is that which promotes increased culture growth. For example with respect to the initiation and growth of Loblolly pine cultures, a desirable pH is between 4.5 and 6, or from 5.5 to 5.8, or about 5.7, or any value subsumed within those ranges.

The invention encompasses the use of any method to maintain desirable pH, including but not limited to the use of buffers in the initiation media, the periodic replacement of old media with fresh media, or the use of one or more metabolites which increase pH as they are metabolized. The amino acid proline is an example of such a metabolite.

In one embodiment, the use of a buffer in media can effectively increase initiation frequency and promote embryogenic culture growth. Compounds having buffering capacity in the range of pH 4-7 are known in the art and include, but are not limited to, those described by Good et al. (Biochemistry, 5, 467,1966), or Ferguson et al. (Ann. Rev. Biochem., 104: 300, 1950). These references are hereby incorporated by reference.

Appropriate buffers include MES (2-(N-morpholino)-ethanesulfonic acid), Bis-Tris (bis[2-hydroxyethyl]-imino-tris[hydroxymethyl]methane; 2-bis[2-hydroxyethyl]amino-2-[hydroxymethyl]-1,3-propanediol), ADA (N-(2-acetamido)₂-iminodiacetic acid), ACES (2-[(2-amino-2-oxoethyl)amino]ethanesulphonic acid), PIPES [piperazine-N,N′-bis(ethanesulphonic acid)], BIS-TRIS-Propane (1,3-bis[tris(hydroxymethyl)methylamino] propane), BES (N,N-bis(2-hydroxyethyl)-2-aminoethanesulphonic acid), MOPS (3-(N-morpholino) propanesulphonic acid), TES (N-tris[hydroxymethyl]methyl-2-amino ethanesulphonic acid) and HEPES (N-2-hydroxyethylpiperazine-N′-2-ethanesulfonic acid).

In one representative example, Example 1, the use of an initiation media containing MES buffer is demonstrated. MES, or any other compound capable of maintaining media pH in the range of pH 4-7, may be advantageously used at a concentration of 10 to 1000 mg/l, 100 to 500 mg/l, 200 to 300 mg/l or any concentration subsumed within these ranges.

Improving Initiation and Embryogenic Culture Growth by Controlling Culture Atmosphere

The inventors have discovered methods of increasing the frequency of embryogenic culture initiation and improving culture growth by. manipulating the atmospheric composition of the culture vessel. “Culture atmosphere” hereafter is defined as the gaseous environment surrounding cultured cells during a particular stage of somatic embryogenesis. Where culturing comprises use of a liquid media, culture atmosphere refers to the gaseous environment above or percolated-through such liquid. In some embodiments, the culture atmosphere. may refer to dissolved gases within a liquid or solid media and may additionally refer to pressure exerted upon the liquid or solid media within a closed chamber.

A “desirable culture atmosphere” is defined as one that improves the efficiency of a particular stage of somatic embryogenesis. For example, in initiation, a desirable culture atmosphere is one that promotes the frequency with which explants initiate cultures compared to explants cultured in ambient atmospheric conditions. Likewise, during maintenance and multiplication, a desirable culture atmosphere is one that increases the growth rate of embryogenic cultures above that in ambient air.

Culture atmosphere can be controlled passively or actively. Cultures controlled passively consist of enclosing culture vessels in a sealed environment so as to avoid or minimize gas loss. Over time, the metabolic activity of the cultured cells changes the atmospheric composition of the culture vessel. The atmospheric composition and its effect on conifer embryogenisis is not well studied. (El Meskaoui, A. and Tremblay, F. M., Plant Cell, Tissue and Organ Culture 56: 201-0.209, 1999). In one experiment, the inventors observed that after three weeks, the oxygen concentration in sealed culture vessels containing growing embryogenic; cultures remained close to 21%, approximately that of ambient air. In contrast, the carbon dioxide concentration in the same vessels appeared to increase significantly, from approximately 350 ppm (ambient air) to about 1200 ppm. The concentration of other gaseous constituents such as ethylene was not measured, but is expected to vary significantly from that of ambient air.

Active control of maintenance culture atmosphere is also possible. Sealed containers holding culture vessels can be provided with an atmosphere of selected composition. In addition to changing the composition of the atmosphere, the atmospheric pressure within the culture vessel can be manipulated. For example, the inventors have cultured embryogenic tissues in a pressure chamber at 1.5, 2.0 and 2.5 atmospheres of pressure. Pressurizing the culture atmosphere subjects tissues to hydrostatic force and additionally increases the partial pressure of various gasses above that of ambient air.

The invention encompasses various methods that provide a desirable culture atmosphere. The mechanisms by which a desirable culture atmosphere enhance embryogenic culture growth are not fully understood. The inventors hypothesize that a desirable culture atmosphere more closely approximates the gaseous environment that early stage embryogenic plant cells experience in nature. Such cells are tightly enclosed within seeds and free gas exchange with the ambient atmosphere is restricted. Indeed, the surrounding seed tissues may provide a selectively permeable barrier for gas exchange. In such an environment, it is likely that CO₂ and volatile hydrocarbons accumulate while oxygen is depleted.

The invention encompasses numerous passive methods of providing a desirable culture atmosphere for the initiation and growth of embryogenic cultures. In one embodiment, cultures are initiated or grown in an enclosed container that is completely sealed from the ambient atmosphere. Although the precise composition of the desirable atmosphere is not completely known, initiation of embryogenic cultures is stimulated in a completely sealed container. In one representative example, multiple culture dishes such as petri dishes or well plates are enclosed together in an airtight container. For example, culture dishes may be sealed in a stainless steel pressure chamber. Alternatively, for example, individual culture vessels may comprise or be enclosed in airtight containers, such as substantially airtight wraps or bags.

In an alternative embodiment, a desirable culture atmosphere is passively supplied by initiating or growing cultures within containers that are. selectively permeable to certain gasses. In one embodiment, cultures are initiated or grown in containers which have a very low permeability to carbon dioxide and oxygen. For example, culture containers can comprise or be enclosed within containers constructed of materials known in the art to have a low carbon dioxide and oxygen permeability. Representative materials include but are not limited to nylon, polyethylene terephthalate (PET), or polyvinyl chloride (PVC).

The invention further encompasses various methods of actively providing a desirable culture atmosphere. A desirable culture atmosphere may be provided by supplying the culture vessel or the area surrounding the culture vessel with, for example, air containing carbon dioxide at concentrations exceeding that of ambient air. Enhanced embryogenic tissue growth in closed containers may partially be due to the increased carbon dioxide concentration which the inventors have observed in such containers (data not shown). A desirable culture atmosphere could be provided by supplying certain gasses either chemically or from an external source, such as a tank of compressed gas.

In a previous pending application (Ser. No. 09/685,338, filed October 11, 2000) herein incorporated by reference, the inventors disclosed that elevated atmospheric pressure can increase conifer embryogenic culture initiation. The present invention extends this previous discovery by demonstrating that elevated pressure also increase the growth rate of previously initiated cultures.

For example, the growth rate of embryogenic cultures can be increased by culturing in a sealed chamber maintained at 1.1 to 5.0, 1.2 to 2.5 or 1.4 to 1.6 atmospheres of pressure. Pressure may be elevated for the majority of the culturing process. Intermittent depressurization of the culturing environment does not affect the efficacy of this method.

Abscisic Acid

The use of abscisic acid (ABA) in culture media is known in the art of conifer somatic embryogenesis during the initiation phase and also during the late developmental and maturation steps. For example, U.S. Pat. Nos. 5,677,185, and 5,856,191, both herein incorporated by reference, disclose that ABA is effective in aiding initiation of Pinus embryogenic cultures. ABA can also be used during later stages of somatic embryogenesis, when early stage somatic embryos are induced to develop into mature planlets. For example, Salajova et al. (1999), working with Pinus nigra, reports the use of ABA in the development media, which induces the early stage somatic embryos to differentiate and mature.

Conversely, the present invention provides novel methods of using ABA for the maintenance and multiplication of previously initiated embryogenic cultures. The use of ABA in the absence of gibberellin during this stage of somatic embryogenesis is not practiced in the art. U.S. Pat. No. 5,294,549 describes the use of gibberellin in the maintenance and multiplication media, and discloses that ABA may be used in conjunction with the gibberellin. However, this disclosure does not suggest that ABA without gibberellin can aid the growth of previously initiated embryogenic cultures. By advocating the use of ABA only in combination with gibberellin, this disclosure teaches away from the use of ABA alone when growing previously initiated cultures. Further, by advocating the use of ABA only in combination with gibberellin, U.S. Pat. No. 5,294,549 clearly teaches away from the use of ABA in combination with an inhibitor of gibberellin during the maintenance and multiplication steps.

The inventors have discovered that ABA alone, or in combination with a gibberellin inhibitor, can promote the growth of previously initiated embryogenic cultures. ABA can promote growth when incorporated into a maintenance and multiplication media at a concentration between 0.001 to 10 mg/l, 0.01 to 1 mg/l, 0.1 to 0.5 mg/l or any concentration subsumed within these ranges. Further, ABA may be effectively used in combination with one or more gibberellin inhibitors, with the gibberellin inhibitor present at a concentration between 0.001 to 500, 0.01 to 50, or 0.1 to 5.0 ppm, or any concentration subsumed within those ranges.

### EXAMPLE 1

### The Beneficial Effects of the Addition of Vitamins and Maintenance of a Desirable pH on Conifer Initiation Frequency and Growth

Beneficial effects on conifer initiation and capture frequencies of the addition of vitamins and maintenance of pH within media has been observed by the inventors. Representative protocols for the enhancement of initiation or. growth in Loblolly pine, Douglas-fir, and Norway Spruce are provided.

Loblolly Pine

Explants were megagametophytes isolated from Loblolly pine summer cones when embryos were at approximately stage 2-4. Seeds were. collected from four individual trees of diverse genotypes. Extracted seeds were soaked in running water for 10 minutes, agitated in 10% liquinox supplemented with 1.1 ml/l of Tween 20 for 10 minutes, and rinsed with water for 30 minutes. Seeds were sterilized in a laminar flow hood by agitating in 20% hydrogen peroxide for 10 minutes and rinsing with sterile deionized water. The seed coat, integuments, and nucellus were removed to isolate the megagametophytes. Culturing was performed in 24-well multi-well culture plates with each well having an approximate volume of 3.0 ml. Two ml of media was poured into each well.

Each replication consisted of eight adjacent wells on a multi-well plate, with one megagametophyte per well, providing three replications per multi-well plate. Twelve replications (for a total of 96 explants) were performed per treatment for each cone collection. The well plates were wrapped in Parafilm®, elastic wrap to minimize evaporation and incubated at 22-24° C. under dark conditions for nine weeks. Initiation frequency was scored for each dish by counting the number of explants which had successfully initiated embryogenic cultures.

The media treatments consisted of: (1) a control media #1043 (see Table 21); (2) media #1218, comprising media #1043 supplemented with 250 mg/l MES buffer; (3) media #1043 supplemented with 0.5 ppm folic acid and 0.05 ppm biotin; and (4) media #1218 supplemented with 0.5 ppm folic acid and 0.05 ppm biotin. As is evident from Table 2, initiation frequency was increased in all treatments relative to the control.

TABLE 2
Initiation frequency with the addition of salts and vitamins

| Treatment | Initiation Frequency (%) |
|---|---|
| Control (Media 1043) | 42.9 |
| +MES | 47.7 |
| +vitamins | 50.3 |
| +MES, +vitamins | 55.4 |

Douglas-Fir

This example demonstrates the effect that buffered media conditions containing vitamins have on somatic initiation, as compared to zygotic initiation. Douglas-fir explants were isolated as above. Previously initiated Douglas-fir cultures were grown in liquid suspension culture media #1133, which comprises media #16, as shown. in Table 21, and the addition of 1.3 ppm ABA. Douglas-fir stage 2 somatic embryos were selected from this liquid maintenance media. A total of three somatic embryo genotypes were tested in each of the four medium types used. For each genotype a total of forty replications was performed. For each replication, a single somatic embryo was placed onto the multiplication medium in the center of each well containing 2 ml medium. The plates containing the embryo were wrapped in Parafilm® and incubated in the dark at 24-26° C. for 4-5 weeks, or until roughly ¾ to 1 cm of growth occurred. The effect the initiation medium had on each genotype was scored measuring colony diameters. Table 3 highlights the effect that ten-fold increases of biotin and folic acid have on initiation in Douglas-fir over the control lacking buffer or vitamins.

TABLE 3
Initiation effect with the addition of salts and vitamins

| | Mean Colony |
|---|---|
| Treatment | Diameter (mm) |
| Control (Media 121) | 5.3 |
| +MES, +1× vitamins (Media 1322) | 5.7 |
| +MES, +10× vitamins (Media 1340) | 6.0 |
| +MES, +100× vitamins (Media 1341) | 6.5 |

The control media used was a Douglas-fir initiation medium known-as media #121. (See Table 21). The remaining test media are comprised of control media #121 but contain 250 mg/l. MES buffer reagent and successive ten-fold increases in vitamins beginning at a concentration of 0.05 mg/l biotin and 0.5 mg/l folic acid. The three test media are media #1322, #1340, and #1341, respectively.

These experiments show that vitamins, such as B-vitamins biotin and folic acid, and controlled pH can be used to substantially improve initiation frequency in conifers. Additionally, the results demonstrate that media constituents may be used effectively in combination, with additive beneficial effects.

Norway Spruce

Mature Norway Spruce seeds were soaked in running water for 1 hour at room temperature and then stored in water for 24 hours in a refrigerator at 4° C. The seeds were sterilized by agitation in 10% liquinox containing 1.1 ml/l Tween 20 for 10 minutes followed by a 30 minute rinse with running tap water. In a sterile laminar flow hood, the rinsed seeds were agitated in 20% H₂O₂ for 10 min., then again rinsed with sterile deionized water for 5 cycles of 5 to 10 min. each. The sterile Norway Spruce seeds were placed into petri dishes onto sterile moistened filter paper for dissection. Seeds were opened and the female gametophyte removed. The female gametophyte was placed on its side and further opened. The embryo was removed from the gametophyte and placed onto a test medium next to the female gametophyte. Petri dishes containing the embryo and gametophyte were covered and wrapped with Parafilm® and incubated in light at 23-24° C. for nine weeks.

The control-medium used for Norway Spruce was media #56. Refer to Table 21 for a detailed listing of media components. The three test media contained combinations of buffering reagent MES and/or vitamins. Test media #1320, in addition to control ingredients, contains 0.25 gm/l MES, 0.05 ppm biotin, and 0.5 ppm folic acid; test media #1362 contains only 0.25 gm/l MES; and test media #1363 contains only 0.05 ppm biotin and 0.5 ppm folic acid.

The results, as shown in Table 4, indicate a increase in initiation frequency for Norway Spruce when using a buffer and vitamins. A slight increase in initiation occurred with the addition of MES buffer alone, while a greater increase in initiation was seen for the addition of vitamins biotin and folic acid, but without the presence of buffer. Not surprisingly, these results differ from responses seen in other conifer. The trend remains, however, that frequency is improved by altering one or several factors in the growth medium.

TABLE 4
Initiation frequency with the addition of salts and vitamins

| Treatment | Initiation Frequency (%) |
|---|---|
| Control (Media 56) | 38.5 |
| +MES (Media 1362) | 40.2 |
| +vitamins (Media 1363) | 45.4 |
| +MES, +vitamins (Media 1320) | 29.6 |

### EXAMPLE 2

### Gibberellin Inhibitors Improve Initiation Frequency

The inventors have observed that exposure of embryos to gibberellin. inhibitors results in improved initiation in conifer.

Loblolly Pine

A representative procedure is shown for Loblolly pine. Experimental procedure follow similarly for other conifer but differ slightly in the composition of media used. The control media used for Loblolly pine was media #1043, as seen in Example 1, supplemented with two different levels of the gibberellin inhibitor paclobutrazol, 0.33 or 1.0 ppm respectively. Explants from four diverse cone collections of Loblolly pine were prepared and cultured as seen in the above Loblolly pine section of Example 1. Here, however, each treatment-genotype test consisted of ten replications of ten explants.

The cultures were covered and incubated in the dark at 24-26° C. for nine weeks. Initiation frequency was measured resulting in roughly a 38% frequency rate for both the control media and media containing 0.33 ppm paclobutrazol. Initiation frequency on media containing 1.0 ppm paclobutrazol, however, averaged greater than 46%. (See Table 5). These results and results obtained for other conifer indicate the effectiveness of gibberellin inhibitors for increasing initiation frequency in conifers.

TABLE 5
Initiation frequency using media with paclobutrazol.

| Treatment | Initiation Frequency (%) |
|---|---|
| Control (Media 1043) | 39.1 |
| +0.33 ppm paclobutrazol | 37.3 |
| +1.0 ppm paclobutrazol | 46.0 |

Douglas-Fir

A further representative example in conifer to demonstrate the effects of gibberellin inhibitors on initiation is Douglas-fir. Four Douglas-fir cone collections with zygotic embryos at stages 2-4 were chosen. Cones were supplied by The Timber Co. from Cottage Grove, Oregon. Seeds were removed from their cones, soaked in running water for 10 min., and agitated in 10% liquinox containing 1.1 ml/l Tween 20 for an additional 10 min. The seeds were then rinsed with running tap water for 30 min. Seeds were transferred into a laminar flow hood where they were sterilized by agitating in 20% H₂O₂ for 10 min. Seeds were rinsed in sterilized water for 5 cycles of 5 to 10 min. each. The sterile seeds were placed into a sterile petri dish and dissected. The dissection was performed by splitting the ovule, gently removing the dominant embryo while leaving the suspensor intact, and placing both the embryo and the connected female gametophyte on the test medium.

The control medium for Douglas-fir, media #121, is listed in Table 21. Test medium #1323 is the same as control media #121 but contains 1.0 ppm paclobutrazol. 2 ml of the appropriate medium was poured into multi-well plates sufficient for 10 replications per cone collection. The plates were incubated in the dark for 8-9 weeks at 24-26° C.

The mean initiation for the control group was 16.4%. The initiation frequency for the four cone collections with the addition of paclobutrazol was determined to be 24.4%. (See Table 6). The observed increase in initiation upon exposure to gibberellin inhibitors is consistent with what we have observed in other conifer.

TABLE 6
Initiation frequency using media with paclobutrazol.

| Treatment | Initiation Frequency (%) |
|---|---|
| Control (Media 121) | 16.4 |
| +1.0 ppm paclobutrazol (Media 1323) | 24.4 |

Norway Spruce

Another example is shown for Norway Spruce. Tissue explants were derived from mature Norway Spruce seeds from freezer storage, originally obtained from F.W. Schumacher Co., Inc., Sandwich, Mass. Seeds were soaked in running water for 1 hour, then let stand in water in a refrigerator for an additional 24 hours. Seed were. then cleaned and sterilized by agitation in 10% liquinox with 1.1 ml/l Tween 20 for 10 min., followed by a tap water rinse for 30 min. Seeds were transferred to a sterile hood and agitated in 20% H₂O₂ for 10 min. Seeds were again rinsed in deionized water 5 times for 5 to 10 min. intervals. After rinsing, seeds were placed in a sterile petri plate onto a sterile moist filter paper for dissection. Seeds were split open and the female gametophyte removed. The female gametophyte was further split and the embryo was removed and placed onto the appropriate control or test medium next to the split female gametophyte. Each petri plate, containing 10 ml of media, was closed, wrapped in Parafilm®, and incubated in light at 23-24° C. for nine weeks.

The control media used for Norway Spruce was media #56, as shown in Table 21. The test medium was comprised of the control media supplemented with the gibberellin inhibitor paclobutrazol at 0.33 ppm. Initiation frequency was determined to be 38.5% for the control media, while the frequency on the control medium with the addition of 0.33 ppm paclobutrazol averaged roughly 48.5%. (See Table 7).

TABLE 7
Initiation frequency using media with paclobutrazol.

| Treatment | Initiation Frequency (%) |
|---|---|
| Control (Media 56) | 38.5 |
| +0.33 ppm paclobutrazol | 48.5 |
| (Media 1360) | |

### EXAMPLE 3

### Gibberellin Inhibitors Increase Initiation Frequency in Buffered Media

The inventors have observed that the pH of explants often drops during the initiation process to levels below those considered desirable. Accordingly, the effect of paclobutrazol, or other gibberellin inhibitors, on initiation in conifer was tested at various concentrations in a media buffered to maintain a desirable pH.

Loblolly Pine

Explants from four Loblolly pine cone collections were prepared and cultured as described in Example 2. The media treatments consisted of control media, #1218, which is media #1043 supplemented. with 250 mg/l MES, and four treatment media, which consist of media #1218 supplemented with 0.33, 1.0, 2.0, and 3.0 ppm paclobutrazol, respectively.

As shown in Table 8, the addition of paclobutrazol to buffered media increased initiation frequency at all concentrations, relative to the buffered control. These results demonstrate that a gibberellin inhibitor such as paclobutrazol can be used to increase initiation frequency in conifers. Additionally, the results demonstrate that paclobutrazol is effective in a media buffered to maintain a desirable pH, which the inventors have shown to be independently effective for increasing initiation. While it appears that a gibberellin inhibitor alone has a greater effect on conifer initiation than buffer alone under the conditions provided, both offer a means to obtain increased embryo multiplication and initiation frequencies in generally recalcitrant Loblolly pine.

TABLE 8
Initiation frequency using media containing MES and paclobutrazol.

| | Initiation |
|---|---|
| Treatment | Frequency (%) |
| Control (Media 1043 + 250 mg/l MES) | 40.5 |
| +0.33 ppm paclobutrazol | 46.8 |
| +1.0 ppm paclobutrazol | 46.0 |
| +2.0 ppm paclobutrazol | 46.1 |
| +3.0 ppm paclobutrazol | 44.7 |

Slash Pine

A further representative example in conifer to show the effects of gibberellin inhibitors in a buffered media is slash pine. Three cone collections with embryos at stages 2-4 were chosen. Removed seeds were soaked in running water for 0.10 min. and then agitated in 10% liquinox containing 1.1 ml/l Tween 20 for an additional 10 min. Seeds were sterilized in a laminar flood by agitating in 20% H₂O₂ for 10 min. Seeds were rinsed in sterilized water for 5 cycles of 5 to 10 min. each. The sterile seeds were placed into a sterile petri dish and dissected. The dissection was performed by splitting the seed coat, removing the integuments and nucellus, and placing the megagametophyte on the test medium.

The control media, media #1369B, is listed in Table 21. Test media #1374 contains the components of media #1369B and 1.0 ppm paclobutrazol. 2 ml of the appropriate media was poured into multi-well plates sufficient for 10 replications for each genotype. The plates were then incubated in the dark for 8 weeks at 24-26° C.

The results in Table 9 indicate that the addition of a gibberellin inhibitor can dramatically improve initiation frequency. The initiation frequency for the control media was observed to be 19.3%, while the frequency observed in media #1374-was 28.5%.

TABLE 9
Initiation frequency using media containing MES and paclobutrazol.

| Treatment | Initiation Frequency (%) |
|---|---|
| Control (Media 1369B) | 19.3 |
| +1.0 ppm paclobutrazol | 28.5 |
| (Media 1374) | |

Douglas-Fir

A further representative example in conifer to demonstrate the effects of gibberellin inhibitors on initiation is Douglas-fir. Four Douglas-fir cone collections with zygotic embryos at stages 2-4 were chosen. Cones were supplied by The Timber Co. from Cottage Grove, Oregon. Seeds were removed from their cones, soaked in running water for 10 min., and agitated in 10% liquinox containing 1.1 ml/l Tween 20 for an additional 10 min. The seeds were then rinsed with running tap water for 30 min. Seeds were transferred into a laminar flow hood where they were sterilized by agitating in 20% H₂O₂ for 10 min. Seeds were rinsed in sterilized water for 5 cycles of 5 to 10 min. each. The sterile seeds were placed into a sterile petri dish and dissected. The dissection was performed by splitting the ovule, gently removing the dominant embryo while leaving the suspensor intact, and placing both the embryo and the connected female gametophyte on the test medium.

The control medium, media #121, is listed in Table 21. Test medium #1383 consists of control media #121 with the addition of 1.0 ppm paclobutrazol, 250 mg/l MES, 5 ppm biotin, 50 ppm folic acid, and 0.1 μM Brassinolide. 2 ml of each medium were poured into multi-well plates sufficient for 10 replications of each cone collection. The plates were incubated in nylon bags with the addition of air for 8-9 weeks at 24-26° C. The mean initiation for the control group was 16.4%. The initiation frequency for the four cone collections with the addition of salts, paclobutrazol, and brassinolide enclosed in a nylon bag was determined to be 38.6%. (See Table 10).

TABLE 10
Initiation frequency using media with paclobutrazol and buffer.

| | Initiation |
|---|---|
| Treatment | Frequency (%) |
| Control (Media 121) | 16.4 |
| +1.0 ppm paclobutrazol + MES + Folic Acid/Biotin + | 38.6 |
| 0.1 μM Brassinolide (Media 1383) | |

### EXAMPLE 4

### Increasing the Growth Rate of Douglas-Fir Embryogenic Cultures Using Gibberellin Inhibitors and Closed Containers

This experiment further illustrates the effect that varying growth conditions can have on conifer development. Specifically this test compares the effect that gibberellin inhibitors have on Douglas-fir initiation to initiation increases resulting from growth in a closed container.

In this example single somatic embryos were used to evaluate growth rate in each test environment. This, therefore, differs from other examples that use zygotic embryos to evaluate effects on initiation or growth. Previous experiments have shown that improved growth measured in somatic initiation tests correlate well with improved natural seed selection initiation (data hot shown). Three Douglas-fir genotypes were selected and were grown in liquid suspension culture media #1133 initially. Media #1133 is comprised of media #16 (see Table 21) and 1.3 ppm ABA. Individual somatic embryos at stage 2 were selected from the maintenance media and placed in the center of petri plates containing either test multiplication medium or control medium. Plates were covered and wrapped with Parafilm®. Embryos were incubated in the dark at 24-26° C. for 4-5 weeks or until about 3/to 1 cm of growth occurs.

Douglas-fir initiation media, media #121, was used as the control media. (See Table 21). Test medium #1323, consisting of the control media plus 1.0 ppm paclobutrazol, was evaluated free from a closed container. Test medium #121.2, remains the same as the control media, but was incubated in a closed nylon bag. The results shown in Table 11 indicate increased growth for both test conditions. The colony diameter for the control was roughly 3.4 mm. The colony diameter for embryos growing on the test medium containing paclobutrazol was approximately 4.4 mm, and the colony diameter for embryos grown in the closed environment were roughly 3.95 mm. This further demonstrates that either factor individually has a stimulatory effect on initiation in Douglas-fir.

TABLE 11
Initiation growth and frequency using media with
paclobutrazol or a closed nylon container.

| Treatment | Initiation growth (mm) |
|---|---|
| Control (Media 121) | 3.4 |
| +1.0 ppm paclobutrazol (Media 1323) | 4.4 |
| +closed nylon bag (Media 121.2) | 3.95 |

### EXAMPLE 5

### Increasing the Growth Rate of Loblolly Pine Embryogenic Cultures Using Gibberellin Inhibitors and Vitamins

Three previously initiated Loblolly pine embryogenic cultures, each representing a different genotype, were grown in liquid media #1133, used as starting material for embryogenic cultures on various treatment media. Using a dissecting scope, individual stage 2 somatic embryos were extracted and placed on treatment media in the center of individual wells on a well plate. Forty embryos were prepared per treatment media, per genotype.

Well plates were wrapped in Parafilm® and cultured under standard conditions at room temperature in the dark for four weeks. Embryogenic cultures derived from the single isolated embryos proliferated and spread over the surface of the culture medium. Spreading cultures were roughly circular in shape, radiating from the central embryo. At the end of four weeks, the growth of each culture was measured by measuring colony diameter using a dissecting scope ocular micrometer.

Five treatment media were compared to control media #1043 (Table 21). Three media consisted of #1043 supplemented with paclobutrazol at concentrations of 0.33, 1.0 and 3.0 ppm. A B-vitamin treatment contained biotin at 0.05 ppm and folic acid at 0.5 ppm.

Growth, as measured by embryogenic tissue diameter on the media surface, was increased relative to the control in the treatments containing paclobutrazol at 1.0 ppm and 3.0 ppm. (See Table 12). Enhanced growth was also observed in the treatment containing folic acid and biotin. These results demonstrate that gibberellin inhibitors, biotin and folic acid may be used to increase the growth rate of embryogenic cultures.

TABLE 12
Growth of embryogenic cultures on media containing
paclobutrazol and B-vitamins.

| | Diameter of tissue | Approximate tissue |
|---|---|---|
| Treatment | (mm) | area (mm²) |
| Control (Media 1043) | 4.01 | 12.6 |
| +0.33 ppm paclobutrazol | 4.13 | 13.4 |
| +1.0 ppm paclobutrazol | 5.00 | 19.6 |
| +3.0 ppm paclobutrazol | 5.23 | 21.5 |
| +0.05 biotin, +.05 folic acid | 5.45 | 23.3 |

### EXAMPLE 6

### Improving the Growth of Loblolly Pine Embryogenic Cultures Using Various Treatments

The survival of embryogenic tissues transferred to liquid multiplication media is highly correlated with the mass of the tissue at the time of transfer. Tissue masses of less than 200 mg have a much lower probability of survival in liquid media than larger masses. This experiment demonstrates that various methods can be used to increase the growth of embryogenic cultures such that a tissue mass of 200 mg is more readily attained.

“Initiates” will be used to refer to recently initiated embryogenic tissues still on the initiation media. In this experiment, initiates were transferred to a maintenance and multiplication media directly from the initiation media. Five treatments, consisting of various media or culture conditions were imposed on the these cultures. Treatments are summarized in Table 13. Treatment media were based on media #1191. (See Table 21.) Initiates derived from eight different cone collections of Loblolly pine were transferred to vials containing 5 ml solid media. Seventy five replications were prepared for each treatment.

TABLE 13
Maintenance culture treatments.

| Treatment # | Media composition/culture conditions |
|---|---|
| 1 | Media 1191 |
| 2 | +1.3 mg/l ABA |
| 3 | +1.3 mg/l ABA/cultured at 1.5 atm |
| 4 | +0.1 μM brassinolide +1.0 ppm paclobutrazol |
| 5 | +1.3 mg/l ABA, +250 mg/l MES, |
| | +0.05 ppm biotin, +0.5 ppm folic acid |

The transferred initiates were grown in the various treatments for four weeks, and thereafter transferred to vials containing fresh media (of the same composition utilized during the previous four weeks). At the end of a second four-week period, cultures were carefully removed from the vials and weighed. The percentage of cultures that had attained a mass of 200 mg or more was calculated for each treatment. Results are summarized in Table 14.

TABLE 14
Percentage of maintenance cultures with mass greater
than 200 mg at eight weeks.

| Treatment # | % mass >200 mg |
|---|---|
| 1- control | 33.7 |
| 2- +ABA | 48.6 |
| 3- +ABA, cultured at 1.5 atm | 61.2 |
| 4- +brassinolide, +paclobutrazol | 45.8 |
| 5- +ABA, +MES, +B-vitamins | 68.4 |

The results demonstrate that numerous methods of the invention can be used to increase maintenance culture growth. For example, in Treatment No. 2, ABA, increased the number of cultures successfully reaching 200 mg within eight weeks. A comparison of Treatments Nos. 2 and 3 demonstrates that culturing at elevated atmospheric pressure increased the growth of the cultures. In Treatment #4, paclobutrazol, successfully enhanced the growth of the cultures. The largest percentage of cultures over 200 mg was obtained in Treatment No. 5, utilizing ABA, MES and the B-vitamins biotin and folic acid. This treatment produced more than twice as many cultures of a desirable size than the control. Comparing Treatments Nos. 5 and 2, the strong growth-enhancing effects of MES and B-vitamins are evident.

These results demonstrate how the invention can be used advantageously in conjunction with a liquid multiplication media, such as that described in Example 7. When embryogenic cultures are transferred to a liquid multiplication media, the frequency with which such cultures survive and proliferate is highly correlated with the tissues' mass. Prior to growth on the maintenance media, the average mass of the initiates in this experiment was only about 30 mg. About 10% of the initiates had a mass of 100 mg or greater, while the majority had a mass of 20 mg or less. Had these initiates been transferred directly from the initiation media to liquid multiplication media, most of the embryogenic cultures would have perished (>75%, based on previous experience). Using the methods of the invention, the majority of initiates could now be grown to a mass of over 200 mg. At that size, a large majority of such cultures (>88%, based on previous experience) can be successfully propagated in a liquid media.

### EXAMPLE 7

### Multiplication of Embryogenic Cultures in a Liquid Media

The following is a description of a liquid multiplication media protocol that may be employed in conjunction with the invention. Initiated embryogenic tissue may be transferred to a liquid multiplication media in order to generate large numbers of somatic embryos. Any liquid multiplication medium known in the art may be used, for example, the liquid media disclosed in U.S. Pat. No. 5,491,090, or liquid media #16, described in Table 21. Initiated cultures are transferred to a culture vessel containing liquid media and agitated by shaking, rolling, stirring, rocking, bubbling or other methods known in the art.

In one embodiment the culture vessel is a 250 ml flat plastic bottle (such as those used in mammalian tissue culture) or a 250 ml Erlenmeyer-style plastic or glass flask. Embryogenic tissue is added to liquid media, for example at a volume of 9 ml of media per gram of initiated tissue. The culture vessel can then be maintained at a favorable temperature, for example 22° C., in a dark room and shaken at about 50 to 100 rpm. Fresh media may be added at regular intervals. For example, by adding or replacing about 1/5^{th} to 1/20^{th} volume of fresh media on a weekly basis.

When a liquid culture is successfully formed, the media will become thick with suspended embryogenic cells. Established cultures may be further multiplied by transferring some of the liquid culture into a container of fresh media. Somatic embryos may be harvested from the liquid by allowing cultures to settle, and pouring off the excess liquid media. Harvested embryos may be cryopreserved or transferred to a development medium and induced to form plantlets using methods known in the art.

### EXAMPLE 8

### Maintaining a Desirable Media pH in Growing Loblolly Pine Embryogenic Cultures Using Buffer

This experiment further demonstrates how the use of buffer in culture media can maintain pH at a more desirable value. The pH of media containing four different concentrations of MES was compared to that of a non-buffered control media during eight weeks of embryogenic culture growth. Maintenance cultures were established on treatment media using single isolated somatic embryos. Maintenance cultures were prepared and cultured essentially as described in Example 5.

The control media was #1043. (See Table 21). The treatment media consisted of #1043 supplemented with 250, 500, 1000 and 1500 mg/l MES. Media pH was adjusted to 5.7 within all treatments prior to autoclaving, pouring and solidification. Immediately after pouring the media, and thereafter at two week intervals, media pH was measured up to the eighth week of culture growth. At each sampling time, four cultures from each media-genotype treatment were destructively sampled to measure the pH of the media directly underlying the growing embryogenic tissue.

Growth of embryogenic cultures acidified the media, decreasing pH in all treatments. In media containing MES, the decrease in pH was less than that of the control. (See Table 15). The results demonstrate that MES can be effectively used to maintain media pH during culturing. Growth of cultures, as measured by culture diameter, was increased, relative to the control, in the media containing 250 mg/l MES. However, in media containing higher concentrations of MES, the growth of embryogenic cultures was inhibited relative to the control. These results suggest that higher concentrations of MES may be inhibitory, despite maintaining a more positive pH.

TABLE 15
Media pH after eight weeks of embryogenic culture growth.

| | Media pH | | | | |
|---|---|---|---|---|---|
| | 0 | 2 | 4 | 6 | 8 |
| Media | weeks | weeks | weeks | weeks | weeks |
| Control (Media 1043) | 5.45 | 5.24 | 4.81 | 4.50 | 4.56 |
| +250 mg/l MES | 5.55 | 5.43 | 4.69 | 4.68 | 4.72 |
| +500 mg/l MES | 5.63 | 5.50 | 4.78 | 4.77 | 4.54 |
| +1000 mg/l MES | 5.67 | 5.57 | 5.38 | 4.70 | 4.89 |
| +1500 mg/l MES | 5.67 | 5.59 | 5.59 | 4.91 | 5.18 |

### EXAMPLE 9

### Increasing the Growth of Loblolly Pine Embryogenic Cultures by Providing a Desirable Culture Atmosphere

This set of experiments using both somatic and zygotic embryos further demonstrate how manipulations in culture atmosphere can increase the growth and initiation rates of embryogenic tissues.

This protocol demonstrates the effect that a closed container environment has on somatic embryos. Maintenance cultures on well plates were prepared from single somatic embryos, essentially as described in Example 5. These maintenance cultures were cultured in three atmospheric treatments. In the control treatment, the Parafilm®-wrapped multi-well plates were placed on shelves in a darkened growth chamber at 24-26° C., standard conditions for growing such maintenance cultures. In the “closed container treatment,” well plates were enclosed in an airtight chamber. In the “pressurized treatment,” well plates were enclosed in an airtight chamber that was pressurized to 1.5 atmospheres. Cultures were grown for five weeks.

Embryogenic cultures in the closed and pressurized treatments grew more than those cultured under standard conditions. (See Table 16). Based on calculations of idealized circular tissue growth, the respective area covered by the cultures was about 45-50% greater in the atmospheric treatments compared to the control. These results demonstrate that providing a desirable culture atmosphere can increase the growth of embryogenic cultures.

TABLE 16
Growth of embryogenic cultures in closed and pressurized containers.

| Treatment | Diameter of tissue | Approximate area |
|---|---|---|
| Control | 2.91 | 6.7 |
| Closed Container | 3.61 | 10.2 |
| Pressurized Container | 3.51 | 9.7 |

This second protocol demonstrates the effect that a closed container environment has on zygotic embryos. Four cone collections with embryos each at stages 2-4 were used to test initiation and frequency. A sufficient number of extracted seeds were soaked in running water for 10 minutes, agitated in 10% liquinox supplemented with 1.1 ml/l of Tween 20 for 10 minutes, and rinsed with running tap water for 30 minutes. Seeds were sterilized in a laminar flow hood by agitating in 20% hydrogen peroxide for 10 minutes and rinsing with sterile deionized water. The seed coat, integuments, and nucellus were removed to isolate the megagametophytes.

The megagametophytes were placed onto solid media #1253 for incubation in the dark for 8-9 weeks at 24-26°. The control embryos were grown in a petri dish, while test samples were grown within a closed metal chamber, which is impermeable to gasses. A comparison of the initiation rate between the control samples and loblolly pine embryos grown in a closed container demonstrate the effectiveness at which a controlled environment further improves growth and initiation. The percent initiation, as seen in Table 17, for embryos incubated in the closed container is approximately 46.6% while the rate in a gas permeable container is roughly 37.4%.

TABLE 17
Initiation frequency of embryogenic cultures in closed container.

| Treatment | Initiation Frequency (%) |
|---|---|
| Control | 37.4 |
| Closed Container | 46.6 |

### EXAMPLE 10

### Decreasing Gas Exchange Stimulates Initiation and Capture in Conifer

The inventors have found conifer embryo initiation and capture stimulated in sealed gaseous environments. Moreover, degrees in stimulation have been observed by the inventors by varying methods of sealing containers, such as with the use of metal chambers, Parafilm® overlays, glass containers, plastic bags, and wrapping tapes.

This protocol demonstrates that improved embryo growth is achieved to varying degrees by incrementally restricting gas exchange of the growth environment. Explants were megagametophytes isolated from Loblolly pine summer cones when embryos were at approximately stages 2-4. Seeds were collected from one cone collection. Extracted seeds were soaked in running water for 10 minutes, agitated in 10% liquinox supplemented with 1.1 ml/l Tween 20 for 10 minutes, and rinsed with water for 30 minutes. Seeds were sterilized in a laminar flow hood by agitating in 20% H₂O₂ for 10 minutes and rinsing with sterile deionized water. The integuments and nucellus were removed to isolate the megagametophytes. The embryos were initiated in media #1253, as described previously, for further transfer to capture medium #1250. (See Table 21).

20 ml glass scintillation vials with threaded lids were prepared as culture containers. Vials were sterilized and filled with 5 ml of sterile test medium, media #1250. In a laminar flow hood, the embryogenic tissue initiates from above was sterilely inserted into the glass vial. Three sets of vials were prepared with varying degrees of gas exchange. These included: 1) a vial with its lid fully tightened without a Paraflilm® wrap; 2) a vial with its lid unscrewed 1/8^{th} turn with a Parafilm® wrap; and 3) a vial with its lid only slightly attached with a Parafilm® wrap. Vials were cultured in the dark for 2 to 2.5 week intervals. After each 2 to 2.5 week incubation, tissue was sterilely extracted, weighed, and placed back into a vial containing fresh media of the same composition for another incubation period under the same conditions. Three incubation periods were performed for a total of six weeks time.

The results shown in Table 18 indicate a correlation between degree of gas exchange and conifer embryo capture. The vial set with the greatest restriction on gas exchange, i.e., the vial with a completely tightened lid, showed the highest percent capture of 51.7%. The vial set with least restriction on gas exchanged had the lowest percent capture of 41.6%. The partially open container yielded an intermediate percent capture of 46.1%. This suggests that a closed container is an appropriate method for capture and embryo multiplication.

TABLE 18
Percent capture of embryogenic cultures in closed and
partially closed containers.

| Treatment Vial | % Capture |
|---|---|
| Fully Closed | 51.7 |
| ⅛^{th} Turn from Closed | 46.1 |
| Mostly Open | 41.6 |

### EXAMPLE 11

### Increasing the Growth Rate of Loblolly Pine Embryogenic Cultures Using Various Treatments

This experiment measured the effects of ABA, vitamins, culturing in a closed container, and culturing at increased atmospheric pressure on the growth of embryogenic cultures. Maintenance cultures were prepared on well plates using single somatic embryos, essentially as described in Example 5. Treatments and growth results after five weeks are summarized in Table 19.

TABLE 19
Maintenance culture treatments

| | | Tissue | Tissue |
|---|---|---|---|
| | Media composition/ | diameter | area |
| Treatment # | culture conditions | (mm) | (mm²) |
| 1 | Control (Media 1191) | 4.11 | 13.3 |
| 2 | +1.3 mg/l ABA | 4.61 | 16.7 |
| 3 | +1.3 mg/l ABA, 250 mg/l MES, | 4.48 | 15.8 |
| | 0.05 ppm biotin, 0.5 ppm folic | | |
| | acid (Media 1250) | | |
| 4 | media #1250 with cultures enclosed | 4.94 | 19.2 |
| | in an airtight container | | |
| 5 | media #1250 with cultures enclosed | 5.13 | 20.7 |
| | in an airtight container at 1.5 | | |
| | atmospheres | | |

All treatments increased the growth of the embryogenic cultures above that of the control. The most growth was observed in Treatments Nos. 4 and 5, where the media constituents of the invention were combined with the invention's atmospheric treatments to obtain substantially greater embryogenic tissue growth.

### EXAMPLE 12

### The Effect of Gibberellin Inhibitors on Zygotic Initiation in Conifer

The inventors have observed that gibberellins, such as gibberellic acid, have an inhibitory effect on initiation frequency in conifer. As such, the inventors considered the effect that corresponding gibberellin inhibitors might have on initiation. This experiment shows a representative protocol for enhancing initiation frequency in Loblolly pine using several different individual gibberellin inhibitors. Specifically, this experiment demonstrates that gibberellin inhibitors with different modes of action, such as paclobutrazol, flurprimidol, chlormequat-Cl, and succinic acid-dh, can increase initiation frequency in conifer.

Four cone collections with embryos at stages 2-4 were used. Eighty explants from each cone collection on each media were tested. Seeds were removed from cones and soaked in running water for 10 min. Seeds were then agitated in 10% liquinox containing 1.1 ml/l Tween 20 for 10 min., and then rinsed with running tap water for 30 min. After transferring to a sterile hood, seeds were agitated in 20% H₂O₂ for 10 min., rinsed with sterile nanopure® H₂O five times for 5 to 10 min. intervals, and then placed into a sterile petri dish for dissection. Seeds were dissected by splitting the seed coat, removing the integuments and nucellus. The megagametophyte was placed onto the appropriate medium. Each multi-well test plate contained 2 ml of solid media. Plates were incubated in the dark for 8 weeks and then scored.

The control media, media #1253, is described in detail in Table 16. All test media, #1275, #1421, #1422, and #1423 were comprised of the control media with the addition of a gibberellin inhibitor. The gibberellin inhibitors and their concentrations were 1.0 ppm paclobutrazol, 0.1062 ppm flurprimidol, 1.58 ppm chlormequat-Cl, and 0.16 ppm succinic acid-dh, respectively.

Results in Table 20 confirm the general trend of increased initiation in conifer when embryo are exposed to gibberellin inhibitors. The inhibitors tested showed slightly different rates of increase in initiation amongst themselves, but resulted in good increases over those embryo grown in conditions lacking an inhibitor. Rates of extrusion changed only slightly between test medium in this study. It is important to note that the salt concentrations used in all medium tested contained two and one half the normal levels typically tested. There is no indication this affected the initiation or extrusion percentages observed.

TABLE 20
Effect of Gibberellin Inhibitors on Initiation Frequency in Loblolly Pine

| | Initiation Frequency | |
|---|---|---|
| Treatment | (%) | Extrusion (%) |
| Control (Media #1253) | 29.5 | 58.6 |
| +1.0 ppm paclobutrazol | 35.3 | 58.1 |
| +0.1062 ppm Flurprimidol | 36.3 | 59.9 |
| +1.58 ppm Chlormequat-Cl | 36.8 | 61.6 |
| +0.16 ppm Succinic Acid-DH | 38.0 | 63.3 |

TABLE 21
Media Compositions

| | Media # (mg/l) | | | | | | | | |
|---|---|---|---|---|---|---|---|---|---|
| Components | 889 | 1043 | 1253 | #16 | 1191 | 1250 | 1369B | 56 | 121 |
| NH₄NO₃ | 200.0 | 200.0 | 200.0 | 603.8 | 603.8 | 603.8 | 825 | — | — |
| KNO₃ | 909.9 | 909.9 | 909.9 | 909.9 | 909.9 | 909.9 | 950 | 50 | 1250 |
| CaCl₂.2H₂O | — | — | — | — | — | — | 110 | — | 200 |
| CaCl₂.6H₂O | — | — | — | — | — | — | — | 220 | — |
| KH₂PO₄ | 136.1 | 136.1 | 136.1 | 136.1 | 136.1 | 136.1 | 170 | 85 | 340 |
| Ca(NO₃)₂.4H₂O | 236.2 | 236.2 | 236.2 | 236.2 | 236.2 | 236.2 | — | — | — |
| MgSO₄.7H₂O | 246.5 | 246.5 | 246.5 | 246.5 | 246.5 | 246.5 | 925 | 160 | 400 |
| Mg(NO₃)₂.6H₂O | 256.5 | 256.5 | 256.5 | 256.5 | 256.5 | 256.5 | — | — | — |
| MgCl₂.6H₂O | 101.7 | 101.7 | 101.7 | 101.7 | 101.7 | 101.7 | — | — | — |
| Kl | 4.15 | 4.15 | 4.15 | 4.15 | 4.15 | 4.15 | 2.075 | 0.145 | 1.0 |
| KCl | — | — | — | — | — | — | | 372.5 | — |
| H₃BO₃ | 15.5 | 15.5 | 15.5 | 15.5 | 15.5 | 15.5 | 15.5 | 3.1 | 5.0 |
| MnSO₄.H₂O | 10.5 | 10.5 | 10.5 | 10.5 | 10.5 | 10.5 | 10.5 | 8.45 | 20.8 |
| ZnSO₄.7H₂O | 14.67 | 14.67 | 14.67 | 14.4 | 14.4 | 14.4 | 21.5 | 4.3 | 8.0 |
| Na₂MoO₄.2H₂O | 0.125 | 0.125 | 0.125 | 0.125 | 0.125 | 0.125 | 0.625 | 0.125 | 0.2 |
| CuSO₄.5H₂O | 0.173 | 0.173 | 0.173 | 0.125 | 0.125 | 0.125 | 0.25 | 0.0125 | 0.024 |
| CoCl₂.6H₂O | 0.125 | 0.125 | 0.125 | 0.125 | 0.125 | 0.125 | 0.065 | 0.0125 | 0.025 |
| FeSO₄.7H₂O | 13.9 | 13.9 | 13.9 | 6.95 | 6.95 | 6.95 | 13.9 | 13.9 | 27.8 |
| Na₂EDTA | 18.65 | 18.65 | 18.65 | 9.33 | 9.33 | 9.33 | 18.65 | 18.65 | 37.3 |
| Maltose | 15K | 15K | 15K | — | — | — | 15K | — | — |
| Sucrose | — | — | — | 30K | 30K | 30K | — | 10K | 15K |
| myo-Inositol | 20K | 20K | 20K | 1K | 1K | 1K | 17.5K | 50 | 1K |
| Casamino Acids | 500 | 500 | 500 | 500 | 500 | 500 | 500 | 500 | 450 |
| L-Glutamine | 450 | 450 | 450 | 450 | 450 | 450 | 450 | 750 | — |
| Thiamine.HCl | 1.0 | 1.0 | 1.0 | 1.0 | 1.0 | 1.0 | 1.0 | 0.05 | 1.0 |
| Pyridoxine.HCL | 0.5 | 0.5 | 0.5 | 0.5 | 0.5 | 0.5 | 0.5 | 0.05 | 0.5 |
| Nicotinic acid | 0.5 | 0.5 | 0.5 | 0.5 | 0.5 | 0.5 | 0.5 | 0.25 | 0.5 |
| Glycine | 2.0 | 2.0 | 2.0 | 2.0 | 2.0 | 2.0 | 2.0 | — | 2.0 |
| L-Aparagine | — | — | — | — | — | — | — | 50 | — |
| NAA | 2.0 | 2.0 | 2.0 | — | — | — | 2.0 | 2.0 | — |
| 2,4-D | — | — | — | 1.1 | 1.1 | 1.1 | — | — | 110 |
| BAP | 0.55 | 0.63 | 0.63 | 0.45 | 0.45 | 0.45 | 0.63 | 1.0 | 45 |
| Kinetin | 0.53 | 0.61 | 0.61 | 0.43 | 0.43 | 0.43 | 0.61 | — | 43 |
| MES | — | — | 250 | — | — | 250 | 250 | — | — |
| Biotin | — | — | 0.05 | — | — | 0.05 | 0.05 | — | — |
| Folic Acid | — | — | 0.5 | — | — | 0.5 | 0.5 | — | — |
| cGMP* | 10 μM | 10 μM | 10 μM | — | — | — | 10 μM | — | — |
| AgNO₃ | 3.398 | 3.398 | 3.398 | — | — | — | 3.398 | — | — |
| Act. Charcoal | 50 | 50 | 50 | — | — | — | 50 | — | 2,500 |
| ABA* | 1.0 | — | — | — | — | 1.33 | — | — | — |
| Brassinolide | — | 0.1 μM | 0.1 μM | — | — | — | 0.1 μM | — | — |
| Gelrite | 2,000 | 2,000 | 2,000 | — | 2,500 | 2,500 | 2,000 | — | 2,000 |
| Difco Agar | — | — | — | — | — | — | — | 7,000 | — |
| Ph titrated to | 5.7 | 5.7 | 5.7 | 5.7 | 5.7 | 5.7 | 5.7 | 5.8 | 5.7 |

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

1-94. (canceled)

95. A method of growing conifer embryogenic cultures comprising culturing explants using a media supplemented with at least one of biotin, folic acid, one or more gibberellin inhibitors, abscisic acid, or a buffer suitable for maintaining a pH of 4.5-6.0.

96. The method of claim 95, wherein the media is supplemented with about 0.001 to 1.0 ppm biotin.

97. The method of claim 95, wherein the media is supplemented with from 0.001 to 1.0 ppm folic acid.

98. The method of claim 95, wherein the buffer is MES.

99. The method of claim 98, wherein the concentration of MES is 10 to 1000 mg/l.

100. The method of claim 95, wherein one or more gibberellin inhibitors are present in the media at a concentration of 0.01 to 10 ppm.

101. The method of claim 100, wherein one gibberellin inhibitor is paclobutrazol.

102. The method of claim 95, wherein paclobutrazol is present in the initiation media at a concentration of 0.01 to 10.0 ppm.

103. The method of claim 95 wherein the abscisic acid is present in a concentration between 0.1 to 1.0 mg/l.

104. The method of claim 95, further comprising culturing the explants in a closed container wherein the free exchange of gases with the ambient atmosphere is fully prevented.

105. The method of claim 95, wherein growing conifer embryogenic cultures comprises initiating conifer embryogenic cultures.

106. The method of claim 95, wherein growing conifer embryogenic cultures comprises using previously initiated conifer embryogenic tissues.

107. A method of growing conifer embryogenic cultures comprising culturing explants using a media supplemented with biotin, folic acid, one or more gibberellin inhibitors, abscisic acid, and a buffer suitable for maintaining a pH of 4.5-6.0.

108. The method of claim 107, wherein the media is supplemented with about 0.001 to 1.0 ppm biotin.

109. The method of claim 107, wherein the media is supplemented with from 0.001 to 1.0 ppm folic acid.

110. The method of claim 107, wherein the buffer is MES in a concentration of 10 to 1000 mg/l.

111. The method of claim 107, wherein one or more gibberellin inhibitors are present in the media at a concentration of 0.01 to 10 ppm.

112. The method of claim 107, wherein the abscisic acid is present in a concentration between 0.1 to 1.0 mg/l.

113. The method of claim 107, wherein growing conifer embryogenic cultures comprises initiating conifer embryogenic cultures.

114. The method of claim 107, wherein growing conifer embryogenic cultures comprises using previously initiated conifer embryogenic tissues.
